(19) United States
(12) Patent Application Publication
(10) Pub. No.: US 2024/0358007 A1
(43) Pub. Date: Oct. 31, 2024

(54) High Efficiency Beef Cattle Having Modified ACTN3 Genes

(71) Applicant: Vincenzo Michael Biella, Bow, NH (US)

(72) Inventor: Vincenzo Michael Biella, Bow, NH (US)

(21) Appl. No.: 18/141,460

(22) Filed: Apr. 30, 2023

(51) Int. Cl. A01K 67/027; C12N 15/113; C12N 15/90

(52) U.S. Cl. CPC A01K 67/0276; C12N 15/113; C12N 15/907; A01K 2217/058; A01K 2217/075; A01K 2227/101; C12N 2310/14

(57) ABSTRACT

A bovine animal or offspring thereof or a bovine cell or gene comprising a modified chromosomal sequence in at least one allele of a gene encoding an Alpha-actinin-3 protein, wherein said modified chromosomal sequence partially or completely impairs the ability of said animal/cell/gene to encode said protein. The bovine retains all previous functionality, but is distinguished by a reduction in the number and cross-sectional area of Type II muscle fibers, and an increase in the proportion of Type I muscle fibers, resulting in improved feedlot efficiency, improved meat quality, and reduced emissions output.

### FIELD OF INVENTION

This invention relates to bovine genetics, specifically it pertains to animals, cells, and genes comprising a modified chromosomal sequence in an ACTN3 gene, as well as methods and materials for the modification of said ACTN3 gene.

### U.S. PATENT DOCUMENTS

This invention references gene modification technologies, such as Clustered Regularly Interspaced Short Palindromic Repeats (CRISPR), RNA Interference, and Transcription activator-like effector nucleases (TALENs), covered by the patents listed herein.

| | | |
|---|---|---|
| U.S. Pat. No. 8,697,359 B1 | April 2015 | Zhang |
| U.S. Pat. No. 8,765,930 B2 | July 2014 | Tuschl, et al. |
| U.S. Pat. No. 9,181,535 B2 | October 2015 | Liu, et al. |

### REFERENCE STATEMENT REGARDING SEQUENCES

The material referenced herein is identical to that in the Sequence Listing XML file.
Reference file name: HEBC_ACTN3_SEQ_1_4
Date of Creation: Apr. 30, 2023
Size of File: 7,827 bytes

### BACKGROUND OF THE INVENTION

The purpose of animal husbandry is to produce animals with a judicious use of resources. Human advancement arose from the first surpluses of ancient farms, and agricultural practices have since focused on qualitative and quantitative improvements in yield.

A focus on efficiency is particularly important with regards to beef cattle (Bos taurus), which are inherently less efficient in conversion of feed to live weight than either the chicken (G.g. domesticus) or pig (S. domesticus).

The Bos taurus ACTN3 gene (Location 29:44584938-44596722) encodes the protein Alpha-actinin-3, which is abundant in, and relegated to, Type II (fast-twitch) skeletal muscle fibers. The ACTN3 gene largely determines the composition of muscle fiber type in the animal at birth, with small variations naturally occurring between different lines of Bos, but with full expression of the gene in all lines.

Beef cattle as produced under human direction specifically for human use, already having undergone centuries of domestication, have no wild equivalent. The Alpha-actinin-3 protein, then, which permits greater force generation at the expense of energy efficiency, has become obsolete. The domestic cow is protected from hypothetical predators by its human caretakers, so a fully functional ACTN3 gene, aside from being redundant, functions only as an economic and environmental burden.

Expression of the Alpha-actinin-3 protein causes the animal to sustain a large proportion of Type II skeletal muscle fibers. These fibers are able to generate energy only at great expense, which contributes to the poor Feed Conversion Ratio (FCR) of beef cattle. During the life of the animal, these inefficient fibers exhibit great contractile force, which in death results in reduced sarcomere length. Meat derived from these fibers is invariably tough, requiring an extensive aging and/or cooking process to become palatable.

Conversely, elimination of the Alpha-actinin-3 protein causes the animal to sustain a large proportion of Type I skeletal muscle fibers. These fibers generate copious energy at relatively low cost, leading to improved FCR. During the life of the animal, these efficient fibers exhibit reduced contractile force, which in death results in increased sarcomere length. Meat derived from these fibers requires less processing to become palatable. More importantly, a greater proportion of a typical beef carcass is made edible, resulting in less waste.

### BRIEF SUMMARY OF THE INVENTION

Bovine animals, offspring thereof, animal cells, and genes that comprise at least one modified chromosomal sequence in a gene encoding an Alpha-actinin-3 protein wherein said modified chromosomal sequence partially or completely impairs the ability of said animal/cell/gene to encode said protein are provided.

Methods of producing animals with at least one modified chromosomal sequence in a gene encoding an Alpha-actinin-3 protein wherein said modified chromosomal sequence partially or completely impairs the ability of said animal/cell/gene to encode said protein are also provided.

The modifications to the chromosomal sequence in a gene encoding an Alpha-actinin-3 protein provided herein reduce the number and cross-sectional area of Type II skeletal muscle fibers, consequentially increasing the proportion of Type I skeletal muscle fibers in the animal at birth, and ensure that such fiber composition is maintained throughout its life. The corresponding alterations in metabolism result in greater feedlot efficiency, improved meat quality, and reduced emissions. We define genetically modified bovine organisms with a modification to such effect as “High Efficiency Beef Cattle” or “HEBC.” Use of said modification in bovine animals used for milk production is within the purview of this invention.

Production of HEBC is preferably accomplished by altering in the animal a chromosomal sequence within the ACTN3 gene which encodes the Alpha-Actinin-3 protein such that said gene is silenced or truncated. By silencing the ACTN3 gene, expression of the Alpha-actinin-3 protein is dramatically reduced or entirely eliminated. By truncating the ACTN3 gene, expression may or may not be hindered, but a shortened or truncated version of the Alpha-actinin-3 protein, with a corresponding reduction in functionality, is produced. In either embodiment, the animal retains complete physical function.

The invention provides any method of modifying a chromosomal sequence in vitro or in vivo as currently practiced in the art for realizing the animal, cell, or gene in this invention.

### BRIEF DESCRIPTION OF THE DRAWINGS

FIG. 1 is a transcript of the wild-type bovine ACTN3 gene, which lists the twenty (20) exons currently known to exist within that gene, and their corresponding nucleotide counts as identified in the ARS-UCD1.2 reference genome.

### BRIEF DESCRIPTION OF SEQUENCES

SEQ ID NO. 1 is the nucleotide sequence comprising the entire wild-type bovine ACTN3 gene as identified in the ARS-UCD1.2 reference genome (DB Identifier: NM_001076157.1).

SEQ ID NO. 2 is the nucleotide sequence comprising exon605694 in the wild-type bovine ACTN3 gene as identified in the ARS-UCD1.2 reference genome.

SEQ ID NO. 3 is the nucleotide sequence comprising exon605694 in the wild-type bovine ACTN3 gene as identified in the ARS-UCLD1.2 reference genome modified by the replacement of the thirty-sixth amino acid (Arginine) with a premature termination codon.

SEQ ID NO. 4 is the nucleotide sequence comprising exon605694 in the wild-type bovine ACTN3 gene as identified in the ARS-UCD1.2 reference genome modified by the deletion of two nucleotides of the sixty-fifth amino acid (Arginine), causing a frameshift mutation.

### DETAILED DESCRIPTION OF THE INVENTION

The invention provides a novel means to simultaneously benefit the consumer, the producer, and the planet which they both inhabit. Reducing or eliminating the expression of the Alpha-actinin-3 protein, or reducing the functionality of said expressed protein results in animals with improved feedlot efficiency, improved meat quality, and reduced emissions.

### Improved Feedlot Efficiency

Quantitative or qualitative reduction or removal of the Alpha-actinin-3 protein within skeletal muscle results in permanent structural and metabolic changes. Research on animal knockout models and humans with two null alleles (R577X, XX genotype) in the ACTN3 gene has demonstrated:
1. A corresponding upregulation of z-disc structural proteins, such as Alpha-actinin-2, which is mediated by the ACTN2 gene (Location: 28:9324680-9403357).
2. Increased anaerobic threshold. “Anaerobic threshold” in this context means the level of activity or degree of muscular exertion, whether measured through acceleration, velocity, force, or strength, in which an organism must switch from aerobic metabolism to anaerobic metabolism.
3. Altered calcineurin signaling supporting a greater than normal shift towards oxidative muscle fiber type in response to any form of physical activity.

Upregulation of the ACTN2 gene results in animals born with a greater proportion of Type I skeletal muscle fibers. The Alpha-actinin-2 protein is ubiquitous in skeletal muscle and interacts with all forms of light and heavy chain myosin.

Conversely, the Alpha-actinin-3 protein preferentially interacts with isoforms of heavy chain myosin, with both proteins being exclusive to Type II (fast twitch) skeletal muscle fibers. A reduction in the expression of these proteins results in a reduction in the cross-sectional area and proportion of Type II skeletal muscle fibers in the animal.

Due to a preponderance of Type I skeletal muscle fibers, as well as an altered physiological response to activity, HEBC have greater mitochondrial density than their wild brethren, and therefore more efficiently utilize aerobic metabolism. These aerobic adaptations result in a greater natural anaerobic threshold. Whereas wild-type cattle use a combination of aerobic and anaerobic metabolism to sustain activity, HEBC primarily utilize aerobic metabolism, resulting in far fewer calories expended for a given quantity of work.

Altered calcineurin signaling ensures that HEBC retain a high proportion of Type I skeletal muscle fibers irrespective of diet, activity, hormone levels, or age. Because the muscles preferentially adapt to physical stimulus by shifting towards aerobic metabolism, the animal never loses its caloric efficiency, and may paradoxically increase its efficiency by engaging in ordinary activity.

By preferentially utilizing aerobic metabolism, HEBC generate most of their Adenosine Triphosphate (ATP) via the citric acid (Krebs) cycle, and conserve a great deal of energy in the process. HEBC utilize the highly inefficient lactic acid (Cori) cycle far less frequently than wild-type cattle, and when utilized, reversion to aerobic metabolism is faster and more thorough. Feedlot efficiency of HEBC necessarily outclasses that of any natural cattle type under ordinary conditions.

HEBC are especially valuable as draught animals. Their distinct muscle physiology provides for improved endurance and increased work capacity. Along with feedlot efficiency, these qualities make them particularly suited for traditional agricultural use and transport in less developed areas.

### Improved Meat Quality

Commercial beef quality in the United States is determined largely by marbling, which is a visual assessment of the intramuscular fat contained in a given cut of beef. The United States Department of Agriculture (USDA) defines the most desirable, or “Prime” beef as having “slightly abundant to abundant marbling” Structural changes resulting in feedlot efficiency, such as fiber-type distribution, also improve meat quality.

By preferentially utilizing aerobic metabolism, HEBC generate most of their ATP via the citric acid (Krebs) cycle working through the mitochondria, using fatty acids as the preferred substrate.

HEBC have a preponderance of Type I skeletal muscle fibers, which naturally store greater levels of intramyocellular lipids within muscle cells, and greater levels of intramuscular fat between muscle cells. Every cut of meat from an HEBC would have more marbling than its unmodified counterpart, because the muscle fibers which comprise the HEBC meat are more likely to have fat stores in close proximity. Every cut of meat from an HEBC is therefore more likely to be judged “Prime” and to be of superior quality, as measured by the USDA, to its unmodified counterpart.

Although taste is subjective, certain quality traits, such as tenderness and redness, are accepted by both producers and consumers as important in determining meat quality.

Type I muscle fibers are more tender, as measured using Warner-Bratzler shear force, largely due to greater sarcomere length and greater denaturation of myosin during heat exposure. The risk of a given cut of meat being “tough” or “stringy” is reduced because those subjective measures are largely tied to the shear force (i.e. mastication) required to disassemble the fibers of which it is comprised.

Type I muscle fibers are colloquially known as “red” muscle fibers given their deep red appearance, which is due to higher levels of myoglobin. The subjective appeal of “red” beef is well established in the food industry, so much so that “Modified Atmosphere Packaging” (MAP) is widely used by beef packaging companies to ensure that beef on display retains its desired color.

HEBC provide a greater proportion of “Prime” meat per carcass than natural beef cattle, while improving both texture and appearance. Waste is further reduced because cuts of meat currently perceived as being of lesser quality, such as the shoulder clod, would become significantly more palatable.

### Reduced Emissions

Cattle are currently the largest contributing livestock species to methane emissions in the United States. The Cattle Enteric Fermentation Model (CEFM), as used by the Environmental Protection Agency (EPA), measures emissions using the equation: DayEmit=[GE×Ym]/[55.65 MJ/kg CH4]. Emission factor equals gross energy intake multiplied by methane conversion rate divided by 55.65.

If other variables remain consistent, emissions increase linearly with gross energy intake. Because HEBC expend energy at a significantly lower rate than wild type cattle, they require less energy intake to support the same weight.

Reduction of food intake necessarily results in less undigested fodder, which leads to less bacterial metabolism and methane production.

### THE INVENTION

The invention provides genetically engineered beef cattle having, within their genome, a modified chromosomal sequence within the ACTN3 gene that impairs or eliminates expression of the Alpha-actinin-3 protein.

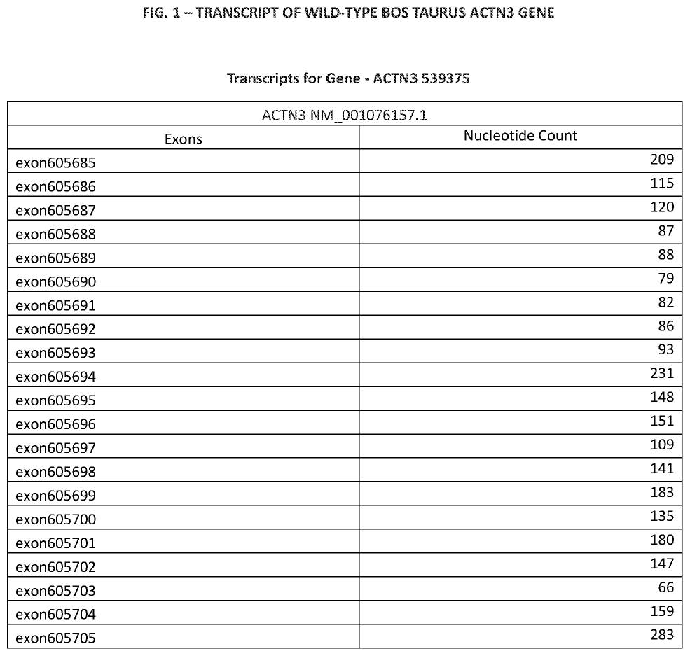

In one embodiment, the modified chromosomal sequence comprises one or more premature termination codon(s) inserted within the ACTN3 gene, preferably within one of twenty available exons (FIG. 1). An example of this embodiment is presented below. Exon 605694 is selected due to its comparatively large size and significant distance from either the initiation or termination of the wild-type gene.

The sequence as naturally occurring in the wild-type gene is illustrated first (SEQ ID NO. 2). The sequence as occurring in the modified gene is illustrated beneath the wild-type gene (SEQ ID NO. 3). The modified sequence replaces the thirty-sixth amino acid—Arginine (GCG)—with a Premature Termination Codon (TAG), resulting in a nonsense mutation.

```
-Exon60594-Wild Type
SEQ ID NO. 2
cta ctg gaa tgg atc cgc cgg acc gtg ccg tgg ctg gag aac cgc gtg
Leu Leu Glu Trp Ile Arg Arg Thr Val Pro Trp Leu Glu Asn Arg Val
  1 5 10 15
ggc gag ccc agc atg agc gcc atg caa cgc aag ctg gaa gac ttc cgg
Gly Glu Pro Ser Met Ser Ala Met Gln Arg Lys Leu Glu Asp Phe Arg
                          20 25 30
gac tac cgg cgc ctg cac aag ccg ccc cgc gtg cag gag aag tgc cag
Asp Tyr Arg Arg Leu His Lys Pro Pro Arg Val Gln Glu Lys Cys Gln
                  35 40 45
ctg gag att aac ttc aac aca ctg cag acc aag ctg cgg ctg agc cac
Leu Glu Ile Asn Phe Asn Thr Leu Gln Thr Lys Leu Arg Leu Thr His
          50 55 60
cgg ccg gcc ttc atg cca tct gag ggc aag ctg gtc tcg
Arg Pro Ala Phe Met Pro Ser Glu Gly Lys Leu Val Ser
  65 70 75 77
-Exon60594-Modified with a nonsense mutation
SEQ ID NO. 3
cta ctg gaa tgg atc cgc cgg acc gtg ccg tgg ctg gag aac cgc gtg
Leu Leu Glu Trp Ile Arg Arg Thr Val Pro Trp Leu Glu Asn Arg Val
  1 5 10 15
ggc gag ccc agc atg agc gcc atg caa cgc aag ctg gaa gac ttc cgg
Gly Glu Pro Ser Met Ser Ala Met Gln Arg Lys Leu Glu Asp Phe Arg
                          20 25 30
gac tac cgg tag ctg cac aag ccg ccc cgc gtg cag gag aag tgc cag
Asp Tyr Arg Leu His Lys Pro Pro Arg Val Gln Glu Lys Cys Gln
                  35 40 45
ctg gag att aac ttc aac aca ctg cag acc aag ctg cgg ctg agc cac
Leu Glu Ile Asn Phe Asn Thr Leu Gln Thr Lys Leu Arg Leu Thr His
          50 55 60
cgg ccg gcc ttc atg cca tct gag ggc aag ctg gtc tcg
Arg Pro Ala Phe Met Pro Ser Glu Gly Lys Leu Val Ser
  65 70 75 77
```

In another embodiment, the modified chromosomal sequence contains one or more frameshift mutation(s) within the ACTN3 gene, preferably within one of twenty available exons. To achieve this, the wild-type sequence is subjected to cleaving and repairs itself via non-homologous end joining. Clustered Regularly Interspaced Short Palindromic Repeats (CRISPR)/Cas9 or Transcription activator-like effector nucleases (TALEN) may be used to achieve a frameshift mutation as presented below. Exon 605694 is selected due to its comparatively large size and significant distance from either initiation or termination of the wild-type gene.

The sequence as naturally occurring in the wild-type gene is illustrated first (SEQ ID NO. 2). The sequence as occurring in the modified gene is illustrated beneath the wild-type gene (SEQ ID NO. 4). The modified sequence presents deletion of the first two nucleotides from the sixty-fifth amino acid—Arginine (CGG), causing the remaining nucleotides to shift, and Alanine (GCC) to be encoded in its place.

```
Exon60594-Wild Type
SEQ ID NO. 2
cta ctg gaa tgg atc cgc cgg acc gtg ccg tgg ctg gag aac cgc gtg
Leu Leu Glu Trp Ile Arg Arg Thr Val Pro Trp Leu Glu Asn Arg Val
  1 5 10 15
ggc gag ccc agc atg agc gcc atg caa cgc aag ctg gaa gac ttc cgg
Gly Glu Pro Ser Met Ser Ala Met Gln Arg Lys Leu Glu Asp Phe Arg
                          20 25 30
gac tac cgg cgc ctg cac aag ccg ccc cgc gtg cag gag aag tgc cag
Asp Tyr Arg Arg Leu His Lys Pro Pro Arg Val Gln Glu Lys Cys Gln
                  35 40 45
ctg gag att aac ttc aac aca ctg cag acc aag ctg cgg ctg agc cac
Leu Glu Ile Asn Phe Asn Thr Leu Gln Thr Lys Leu Arg Leu Thr His
          50 55 60
cgg ccg gcc ttc atg cca tct gag ggc aag ctg gtc tcg
Arg Pro Ala Phe Met Pro Ser Glu Gly Lys Leu Val Ser
  65 70 75 77
-Exon60594-modified with a frameshift mutation
SEQ ID NO. 4
cta ctg gaa tgg atc cgc cgg acc gtg ccg tgg ctg gag aac cgc gtg
Leu Leu Glu Trp Ile Arg Arg Thr Val Pro Trp Leu Glu Asn Arg Val
  1 5 10 15
ggc gag ccc agc atg agc gcc atg caa cgc aag ctg gaa gac ttc cgg
Gly Glu Pro Ser Met Ser Ala Met Gln Arg Lys Leu Glu Asp Phe Arg
                          20 25 30
gac tac cgg cgc ctg cac aag ccg ccc cgc gtg cag gag aag tgc cag
Asp Tyr Arg Arg Leu His Lys Pro Pro Arg Val Gln Glu Lys Cys Gln
                  35 40 45
ctg gag att aac ttc aac aca ctg cag acc aag ctg cgg ctg agc cac
Leu Glu Ile Asn Phe Asn Thr Leu Gln Thr Lys Leu Arg Leu Thr His
          50 55 60
gcc ggc ctt cat gcc atc tga ggg caa gct ggt ctc g
Ala Gly Leu His Ala Iso Gly Gln Ala Gly Leu
  65 70 75 76
```

In another embodiment, the modified chromosomal sequence comprises one or more missense mutation(s).

In another embodiment, the modified chromosomal sequence comprises genetic material obtained from a different organism.

In another embodiment, the modified chromosomal sequence comprises one or more DNA template(s).

In another embodiment, the modified chromosomal sequence comprises genetic material introduced using adenoviral, lentiviral, or other viral vectors.

### Methods and Materials

The invention provides any method of modifying a chromosomal sequence in vitro or in vivo as currently practiced in the art for realizing the animal, cell, or gene in this invention.

In the preferred embodiment, the invention uses the Clustered Regularly Interspaced Short Palindromic Repeats (CRISPR) system to edit the target gene genomic DNA in order to introduce a disabling mutation. This is the best mode contemplated by the author for carrying out the invention. As currently practiced in the art, the CRISPR system consists of an enzyme which is an endonuclease capable of generating DNA strand breaks in an RNA-targeted manner. Currently, Cas9 and derivatives thereof is the most commonly used form of the enzyme, but related enzymes, naturally occurring or artificially engineered in whole or in part, may also be used.

In this embodiment, the chosen CRISPR enzyme is introduced into the target cells along with a small guide RNA (sgRNA) which encodes a few key sequences. The first is typically a 17-24 nucleotide sequence which is in whole or in-part reverse complementary to one of the two strands of gene genomic DNA being targeted. For this example, we use a 20 nucleotide sequence (20mer). This 20mer is designed in such a way as to be targeted at a specific exon of the gene, and if using Cas9 or other Cas9 variants requires that the Protospacer Adjacent Motif (PAM) matching “NGG”—where N represents any of the four DNA nucleotides—is directly adjacent to the 3′ end of the 20mer targeting sequence. This “NGG” sequence should also be reverse complementary to the gene genomic DNA as well as encoded on the 3′ end of the 20mer RNA in the reverse complement; necessitating the identification of a suitable 23mer gene genomic sequence as the “NGG” motif must be present. In this embodiment the targeting sequence may vary in length and does not strictly have to be a 20mer.

The PAM sequence may or may not be required or may vary in sequence as dictated by the corresponding CRISPR enzyme chosen to implement this embodiment. If using the Cas9 enzyme or derivatives a Guide RNA (gRNA) scaffold sequence is also required to be located directly on the 3′ end of the 23mer targeting sequence so that the Single Guide RNA (sgRNA) is recognizable and usable by the Cas9 enzyme. The requirements and design of this scaffold sequence may change as the art progresses and/or if enzymes alternative to Cas9 and derivatives are used. In this preferred embodiment both the Cas9 or related enzyme and the sgRNA cassette, which in this example comprises 23mer and appropriate scaffold, is introduced into target cells by either lentiviral or adenoviral vectors.

This may be an “all-in-one” vector design where DNA encoding the CRISPR enzyme is expressed from a promoter such as Simian Virus 40 (SV40), Cytomegalovirus (CMV), Phosphoglycerate Kinase (PGK) or any other RNA polymerase II compatible promoter found in the art along with a DNA cassette encoding the sgRNA/scaffold driven by a H1, U6 or any other RNA polymerase III compatible promoter. Different viruses may be used to deliver individual components, such as one virus used for Cas9 and another used for the sgRNA.

The DNA encoding Cas9 and the sgRNA may also be delivered into target cells by transient transfection of plasmid DNA using either chemical methods or electroporation as defined in the art. Another implementation comprises the transfection of in vitro transcribed and purified sgRNA and Cas9 mRNA by either chemical means or electroporation into target cells.

This embodiment may also be implemented by using cells that already stably or inducibly express Cas9 and then introducing only the sgRNA by either transient transfection in either DNA or RNA purified form. Cells that already stably or inducibly express Cas9 may also be further transduced with an adenoviral or lentiviral vector that encodes the sgRNA as described.

If working with animal zygotes or embryos another implementation would be to microinject in vitro transcribed and purified Cas9 mRNA and sgRNA directly into the zygotes or embryos. However it is achieved, it is currently understood in the art that the introduction of Cas9 and an appropriately-designed sgRNA results in the production of double strand DNA breaks at the target site that are subsequently repaired primarily by the non-homologous end joining (NHEJ) DNA repair process. In so doing insertions, deletions and/or substitutions may occur in one or both alleles of the gene that disrupt the ability of the gene to produce its wildtype RNA and ultimately wild-type protein products.

In another embodiment, the invention uses CRISPR as previously described along with a DNA template complementary to the target site that introduces defined mutations in the target gene. The introduction of an appropriately-designed template as defined in the art, containing homology arms located 5′ and 3′ of the sgRNA target site, engages the homology directed repair (HDR) cellular mechanism to incorporate the template into the genomic DNA when the double strand break is created by the Cas9 or other appropriate enzyme. This template may introduce in-frame stop codons, insertions, deletions or base substitutions that disrupt the production of or alter the nature of the target gene. This template may be introduced by either transient transfection methods, such as chemical or electroporation, or by lentiviral or adenoviral delivery.

In another embodiment, the invention uses CRISPR as previously described with two key changes. The first change is the replacement of Cas9 with a modified form of Cas9 that only cuts one strand of DNA rather than both, known to those skilled in the art as a “nickase”—examples being D10A or H840A mutated Cas9, or with any other enzyme that is localized to the genomic target and can perform this function. The second change is the use of two sgRNAs (rather than one) which are designed against the target genomic sequence in such a way that one sgRNA is reverse complementary to the top strand and the other is reverse complementary to the bottom strand. In this implementation the combination of the two sgRNAs and the nickase causes a single-strand cut to be made on both strands. When these cuts are in relatively close proximity, preferably no more than 100 nucleotides apart, efficient double-stranded NHEJ-directed DNA repair can occur between the single-strand cuts. In this embodiment the target sequence of the gene is located between the sgRNAs. The genomic target sequence may be exonic, intronic, a regulatory region of the gene or a combination thereof. In one preferred implementation the target region is an exonic splice junction (either acceptor or donor) with one sgRNA located in the exon and the other sgRNA located in the adjacent intron no more than 50 base pairs apart. This is referred to in the art as Splice Junction-targeted CRISPR (spJCRISPR).

The delivery methods of the modified Cas9 or other appropriate enzyme and the two sgRNAs to target cells may be achieved in all the ways previously described. Upon NHEJ-mediated DNA repair between the single-strand breaks, the splice junction is eliminated, leading to the retention of the intron during mRNA processing. This intronic retention is expected to lead to the nonsense-mediated decay (NMD) of the entire target gene transcript; preventing it from being translated into its wild-type protein(s).

In another embodiment, the invention uses CRISPR as previously described in combination with the template-directed DNA repair strategy. This allows for the template-directed repair of the genomic target sequence located between the two sgRNAs.

In another embodiment, the invention uses CRISPR Prime Editing (PE) to edit the genomic sequence of the target gene. Here, a nickase form of Cas9, such as D10A or H840A, or other appropriate enzyme is fused to a reverse transcriptase on the c-terminus and introduced into target cells along with a Prime Editing Guide RNA (pegRNA). The pegRNA is engineered to contain a guide RNA sequence designed against a target site in the genomic DNA of the target gene and is fused with a repair template. Upon nicking of the target strand, a 3′ flap is created which is used as a primer by the pegRNA to prime reverse transcription of the pegRNA template. This reverse transcription produces a single-strand of DNA which is incorporated into the nicked strand of DNA to replace the target sequence. Typically, the pegRNA template introduces single-base pair substitutions or small insertions/deletions which may alter or disrupt gene function. Mismatch repair is utilized by the cell to introduce the corresponding mutations on the complementary strand, avoiding the need to engage the NHEJ or HDR DNA repair pathways to achieve these genomic edits.

To modify the target gene, PE may be used in one implementation to introduce an in-frame stop codon causing premature translational termination of the protein product. PE may also be used to create a defined insertion/deletion that would cause a frameshift mutation, again disrupting mRNA translation. The nickase Cas9 and pegRNA may be delivered by any means as previously outlined.

In another embodiment, the invention uses CRISPR systems that utilize a catalytically deficient (dCas9) or Cas9 nickase (Cas9n) fused to either cytosine or adenine deaminases, such as Apolipoprotein B mRNA Editing Enzyme Catalytic Polypeptide 1 (APOBEC1) and Adenine Base Editor 7.10 (ABE7.10), respectively, as well as other supporting proteins where necessary to alter the target gene genomic DNA. There are two primary classes of CRISPR base editors—Cytosine Base Editors (CBEs) and Adenine Base Editors (ABEs). CBEs effect cytosine to thymine (represented by uracil, C→T) conversions while ABEs convert adenine to guanine (represented by inosine, A→G). In this invention base editors may be deployed to alter the target gene coding sequence by introduction of disabling mutations or premature termination codons. The base editors are introduced into target cells in combination with a sgRNA that targets the sequence to be modified. All of the methods previously described may be applied in this embodiment to achieve enzyme and sgRNA delivery.

In another embodiment, the invention uses CRISPR systems that utilize a catalytically deficient (dCas9) fused to either a transcriptional transactivator (CRISPRa) or transcriptional repressor (CRISPRi) domain. Transactivating domains of proteins such as VP64, p65 and GCN4 may be fused to dCas9 or another suitable sgRNA-targeted enzyme to create artificial sgRNA-targeted transcriptional activators. This CRISPRa protein may then be overexpressed along with a sgRNA that targets the promoter or other regulatory region of the target gene to increase the expression of the target gene. In another implementation, transcriptional repressor domains, such as the Kruppel Associated Box (KRAB) domain, may be fused to dCas9 or another suitable sgRNA-targeted enzyme to create an artificial repressor or CRISPRi protein. This CRISPRi protein may then be overexpressed along with a sgRNA that targets the promoter or other regulatory region of the target gene to decrease the expression of the target gene. In another implementation multiple sgRNAs may be used in conjunction with either the CRISPRa or CRISPRi proteins to achieve the desired level of target gene expression. All of the methods previously described may be applied in this embodiment to achieve fusion protein and sgRNA delivery.

In another embodiment, the invention uses RNA interference (RNA) to suppress expression of the target gene from the level of its mRNA. Specifically, this is achieved by overexpression of a short hairpin RNA (shRNA). shRNAs are small RNAs designed to fold into a stem-loop structure. Typically, a 21 nucleotide (21mer) targeting sequence that is complementary to the target mRNA is located immediately 5′ of the loop sequence “CTCGAG.” Immediately 3′ of the loop sequence is the reverse complement of the 21mer targeting sequence, which is then immediately followed by a RNA polymerase III transcriptional terminator sequence “TTTTT.” This small RNA is typically expressed in cells from a RNA polymerase III driven promoter such as U6 or H1. When overexpressed inside cells, the loop is cleaved by the Dicer enzyme, creating a double-stranded (dsRNA) duplex. Following strand separation each single-strand of RNA is then loaded into the RNA-Induced Silencing Complex (RISC). The RISC scans mRNAs using the single-strand RNA as a guide. When the single-strand RNA hybridizes to its mRNA, target mRNA target cleavage is engaged by the Argonaute RISC Catalytic Component 2 (AGO2) protein. This cleavage renders the target mRNA unable to be translated, ultimately resulting in functional gene inhibition. In this embodiment shRNAs are preferably delivered by lentiviral or adenoviral vectors into the target cells, zygotes or embryos in order to achieve stable and long lasting shRNA overexpression. This stable shRNA overexpression then results in stable target gene silencing.

In another embodiment, the invention uses Transcription activator-like effector nucleases (TALEN) to disrupt the genomic DNA sequence of the target gene. In a preferred implementation, individual TALEs are designed to bind DNA located 5′ and 3′ of genomic target gene exonic sequence. Each TALE is fused with a Fok1 endonuclease which only creates double-strand DNA breaks as a dimer. This Fok1 dimerization requirement therefore necessitates that the TALEs bind in close proximity to each other, as designed to do by their predetermined DNA sequence specificity 5′ and 3′ of the genomic target site. After cleavage by Fok1, the NHEJ DNA repair pathway creates insertions, deletions, and/or substitutions within the targeted genomic sequence, disrupting target gene function.

To avoid the somewhat unpredictable nature of NHEJ. HDR may be induced by providing an appropriate DNA template complementary to the target DNA. This repair template may then contain defined mutations that introduce stop codons, insertions, deletions or other specific base pair mutations to alter gene function or ultimately disrupt gene expression. The individual TALEs and optional repair template can be introduced into target cells, zygotes or embryos using all the approaches previously outlined.

In another embodiment, the invention uses RNAi methodology as would otherwise be practiced by people skilled in the art.

In another embodiment, the invention uses Clustered Regularly Interspaced Short Palindromic Repeats (CRISPR)/Cas9 methodology as would otherwise be practiced by people skilled in the art.

In another embodiment, the invention uses Transcriptor activator-like effector nucleases (TALEN) methodology as would otherwise be practiced by people skilled in the art.

1. A bovine animal or offspring thereof or a bovine cell or gene comprising a modified chromosomal sequence in at least one allele of a gene encoding an Alpha-actinin-3 protein, wherein said modified chromosomal sequence partially or completely impairs the ability of said animal/cell/gene to encode said protein.

2. The animal, cell, or gene of claim 1, wherein said modified chromosomal sequence comprises a premature termination codon or nonsense mutation.

3. The animal, cell, or gene of claim 1, wherein said modified chromosomal sequence comprises a missense mutation.

4. The animal, cell, or gene of claim 1, wherein said modified chromosomal sequence comprises genetic material from a different organism.

5. The animal, cell, or gene of claim 1, wherein said modified chromosomal sequence comprises cleaving and/or non-homologous end joining.

6. The animal, cell, or gene of claim 1, wherein said modified chromosomal sequence comprises enzymatic or physical modulation.

7. The animal, cell, or gene of claim 1, wherein said modified chromosomal sequence comprises adenoviral, lentiviral, or other viral vectors.

8. The animal, cell, or gene of claim 1, wherein said modified chromosomal sequence comprises homology directed repair.

9. A method of producing a modified bovine animal, cell, or gene comprising:
a) Obtaining a bovine cell, wherein the genomic DNA of the cell preferably comprises a wild-type ACTN3 gene;
b) Altering the chromosomal sequence of said gene such that the ability of that gene to encode the Alpha-actinin-3 protein is partially or completely impaired.

10. The method of claim 9 wherein step b is completed via the introduction of a premature termination codon into any location within the ACTN3 gene.

11. The method of claim 9, wherein step b is completed via the introduction of genetic material from another organism into any location within the ACTN3 gene.

12. The method of claim 9 wherein step b is completed via a gene modification system which cleaves the DNA within the ACTN3 gene, such as Clustered Regularly Interspaced Short Palindromic Repeats (CRISPR)/Cas9 or Transcription activator-like effector nucleases (TALEN).

13. The method of claim 9, wherein step b is completed via a gene modification system which disrupts the DNA within the ACTN3 gene such that non-homologous end joining is induced.

14. The method of claim 9, wherein step b is completed via the replacement, transference, deletion, or addition of one or more nucleotides within the ACTN3 gene.

15. The method of claim 9 wherein step b is completed via a gene modification system which results in enzymatic or physical modulation of mRNA within the ACTN3 gene, such as RNA interference (RNAi).

16. The method of claim 9 wherein step b is completed using an adenoviral, lentiviral, or other viral vector.

17. The method of claim 9 wherein step b is completed via induction of homology directed repair.


SEQUENCE LISTING (4 sequences)

SEQ ID NO: 1 - DNA, 2892 bp, Bos taurus
  source 1..2892: mol_type = genomic DNA
cttaacgcggcccccgtgttctggagcgcgccgagcagagcggagccaggagcccgaacaagatgatgatggttctgcagcccgagggtctggggaccggggaggggcccttcgcgggcggccgtgggggcggcgaatacatggaacaggaggaggactgggaccgcgacctgctgttggacccggcctgggagaagcagcagcggaaaaccttcactgcctggtgcaactcacacctgcgcaaggctggcacccagatcgagaacatcgaggaggatttccgcaatggcctcaaactcatgttgctcctggaggttatttcaggagagaggctgcccagaccagacaaaggcaagatgcgcttccacaagattgccaatgtcaacaaggccctggatttcattgccagcaagggggtgaagctggtgtccatcggtgctgaagagattgttgacgggaacctgaagatgaccctgggcatgatctggaccatcatccttcgctttgccatccaggacatctccgtggaagaaacctcggccaaggaaggcttgctcctgtggtgtcagcggaagacggcgccgtaccgcaacgtcaacgtgcagaacttccacaccagctggaaggatgggctggccctctgtgctctcatccaccgacaccgccccgacctcattgactatgccaaactgcgcaaggacgaccccatcggcaacttgaacaccgcctttgaggtggccgagaagtacctggacatccctaagatgctggatgcagaagacattgtgaacaccccaaagcctgatgagaaggccatcatgacctatgtctcctgcttctaccacgcctttgccggggcggagcaggcagagacagctgccaacaggatctgcaaagtgctggccgtgaaccaggagaacgagaagctgatggaggaatatgaaaagctggccagtgagctactggaatggatccgccggaccgtgccgtggctggagaaccgcgtgggcgagcccagcatgagcgccatgcaacgcaagctggaagacttccgggactaccggcgcctgcacaagccgccccgcgtgcaggagaagtgccagctggagattaacttcaacacactgcagaccaagctgcggctgagccaccggccggccttcatgccatctgagggcaagctggtctcggacatcgccaacgcatggcggggcctggagcaggcagagaagggctatgaggattggctgctctccgagatccggcgcctacagcggctgcagcacctggcggagaagttccagcagaaggcctccctgcatgaagcctggacccggggaaaggaggacatgctgagccagagggactacgagacggcttcgctgcaggaggtgcgggccttgctgcggcgccacgaggccttcgagagcgacctggcggcgcaccaggaccgcgtggagcacatcgctgcgctggcccaggagctcaatgagctggactaccacgaggcagcctcggtgaacagccgctgccaggccatctgtgaccagtgggacaacctgggcacgctgacccagaagaggcgggatgcactggagcggatggagaagctcctagagaccatcgatcagctacagctggagtttgcccggcgggcagcgcccttcaacaactggctggacggcgccgtggaggacctgcaggacgtgtggctggtgcactcggtggaggagacccagagcttggtgacagcacacgaccagttcaaggcaacactgcccgaggccgaccgagagaggggtgccatcctgggcatccagggtgagatccagaagatctgccaaacgtacggactgcggcccagctccaccaacccctacatcaccctcaccccgcaggacatcaacaccaagtgggacacggtccgaaagctggtacccagccgtgaccagatgctgcaggaggagctgacgcggcagcaggtgaacgagaggctccggcggcagtttgcagcccaggccaacgccatcgggccctggatccaggggaaggtggaggaagtggggcgcctagccgcggggatggccggctctctggaggagcagatggcgggcctgcggcagcaggagcagaatatcatcaactacaagagcaacatcgaccggctggagggagaccaccagctgctgcaggagagtctggtgtttgacaacaagcacacggtctacagcatggagcacatccgcgtgggctgggagcagctgctcacctccatcgcacgcaccatcaacgaggtggagaaccaggtactgacccgagacgccaagggcctgagtcaggagcagctcaacgagttccgggcatctttcaaccactttgaccggaagcggaatggcatgatggagcctgatgacttccgggcctgcctcatctccatgggctatgacctgggggaggtggagttcgctcgcatcatgaccatggtggaccccaacgcagctggggttgtgaccttccaagccttcatcgactttatgacccgagagacagccgagacagacacggctgagcaggttgtggcctcgttcaagatcctggcgggagacaagaactacatcaccgccgaggagctgcggcgggaactcccggcggagcaggccgagtactgcatccgccgcatggcgccctacaagggggctggggctccagctggagccctggactacgtggccttctccagtgccctctacggggagagtgacctctgacgttgaccccagagcttccctactcaagtccggagagagggtgctccctctggctgatcccacccatcccttggagccatggccacagagccgagtgtttctcaataaagatccatcgtgggcc

SEQ ID NO: 2 - DNA, 231 bp, Bos taurus
  source 1..231: mol_type = genomic DNA
ctactggaatggatccgccggaccgtgccgtggctggagaaccgcgtgggcgagcccagcatgagcgccatgcaacgcaagctggaagacttccgggactaccggcgcctgcacaagccgccccgcgtgcaggagaagtgccagctggagattaacttcaacacactgcagaccaagctgcggctgagccaccggccggccttcatgccatctgagggcaagctggtctcg

SEQ ID NO: 3 - DNA, 231 bp, Bos taurus
  source 1..231: mol_type = genomic DNA
ctactggaatggatccgccggaccgtgccgtggctggagaaccgcgtgggcgagcccagcatgagcgccatgcaacgcaagctggaagacttccgggactaccggtagctgcacaagccgccccgcgtgcaggagaagtgccagctggagattaacttcaacacactgcagaccaagctgcggctgagccaccggccggccttcatgccatctgagggcaagctggtctcg

SEQ ID NO: 4 - DNA, 229 bp, Bos taurus
  source 1..229: mol_type = genomic DNA
ctactggaatggatccgccggaccgtgccgtggctggagaaccgcgtgggcgagcccagcatgagcgccatgcaacgcaagctggaagacttccgggactaccggcgcctgcacaagccgccccgcgtgcaggagaagtgccagctggagattaacttcaacacactgcagaccaagctgcggctgagccacgccggccttcatgccatctgagggcaagctggtctcg